(19) United States
(12) Patent Application Publication
(10) Pub. No.: US 2003/0186463 A1
(43) Pub. Date: Oct. 2, 2003

(54) Method for clearing color and debris from or adding adjuvants or reactants to a selected portion of a chromatographic strip alone or in combination with a cell lysing step

(76) Inventors: Robert Hudak, Carlsbad, CA (US); Roger N. Piasio, Cumberland Foreside, ME (US)

Correspondence Address: The M.H. Sears Law Firm, Chartered, 910 Seventeenth Street N.W., Washington, DC, 20006, US

(21) Appl. No.: 10/098,300

(22) Filed: Mar. 18, 2002

(51) Int. Cl. G01N 33/558

(52) U.S. Cl. 436/514

(57) ABSTRACT

In an improved method for clearing a field of observation or treatment of unwanted color or other extraneous material, a chromatographic strip of paper, plastic, glass or like material, with or without simultaneous deposit of adjuvant or reactants on the cleared field, the strip is immersed to a level just barely above the juncture of the zone to be cleared with the preceding zone of the strip in a chase liquid, such as water containing adjuvant if desired. Where red blood cells containing parasitic pathogens such as cholera parasites are to be assayed, a lysing step is performed on the same strip prior to the clearing strip.

### INTRODUCTION

The present invention relates to an improved method for clearing a field of observation or treatment of unwanted color and other extraneous material that is rapid and exceptionally efficacious. The method is applicable to a wide variety of assays and to other laboratory operations including chemical, biochemical and biological reactions that lend themselves to being conducted, at least for purposes of removing color or other unwanted materials that may obscure a background field of vision, on a chromatographic test strip comprised at least in part of paper, nitrocellulose, nylon, polyester, glass or like materials. The invention is also useful in depositing adjuvants or reactants on a field on which clearance of unwanted color or debris is being, or has been, effected.

### BACKGROUND OF THE INVENTION

Many parasitic pathogens that commonly infect humans and other mammals preferentially invade red blood cells, making it necessary to design assays for detecting their presence in a blood sample by means of an assay process that includes a lysing step which breaks open these cells and exposes any parasitic agent contained therein to the action of reagents needed to identify it or to treat it experimentally. Among parasitic diseases are many of those indigenous to tropical areas, such as cholera, dengue fever, filariasis, Ebola fever, Marburg fever, etc. These diseases, which affect humans, are severe and the development of more rapid and reliable tests for early diagnosis of each is an ongoing effort. A parasitically caused disease that tends to affect dogs and related mammals is canine heartworm. In all such situations, the ability to lyse red blood cells (i.e., split them open) and thereafter immediately and rapidly clear the field of vision and further reaction of irrelevant debris and the intense red color of hemoglobin is important.

In addition to red blood cells, other types of cells that may need to be lysed, followed by rapid clearing of biological debris in order to permit detection of an infective organism on a rapid and accurate basis include white cells, virions, phages, micelles, liposomes and other structures similar in that they each have an internal and an external region separated by a rupturable boundary.

Heretofore, the diagnosis of parasitological disease states has most reliably been determined by culturing the suspected parasite and subjecting the culture after a period of days to microscopic examination. Because these parasite-caused diseases are extremely contagious, culturing of blood samples containing the parasite often poses serious danger to laboratory personnel. For example, Ebola and Marburg viruses, both of which cause hemorrhagic fevers lethal to about 80% of their mammalian (including human) victims are extremely dangerous to laboratory personnel. A Mar. 4, 2002 Wall Street Journal article (pages B1 and B4)reports that scientists must presently wear space suits and work behind air-locked doors in high containment Biosafety Level 4 laboratories when working with live Ebola virus. Access to a rapid, effective way of clearing a working field of hemoglobin and cellular debris might be of great aid in enabling the development of at least one rapid test for detecting the presence of the virus in humans or other mammals and might equally serve to make the test practical to use.

Efforts to use mammalian blood as a sample for various diagnostic tests have always posed problems of how best to clear the field of operation of red color so as to permit observation of end points, etc. Normally these efforts involved multiple washings, and/or “reverse” washings, but seldom, if ever, has it been possible to remove all color from the field of operation by this means.

Similarly, efforts to remove unwanted debris, even in the absence of unwanted color, from a field on which cells have been lysed have frequently been less than totally successful, because the only way of proceeding usually involved multiple washing and/or “reverse” washing steps which only partially took away the debris.

### BRIEF DESCRIPTION OF THE INVENTION

This invention is practiced by transferring to a chromatographic strip all or a part of the operation of a color-producing or debris-producing operation. The strip may initially be dry or it may have been wetted at least partially by other-liquid operations. For example, cells suspected of containing a parasite may have been lysed upon the strip. The strip may comprise several pads of different material joined together-or, alternatively it may comprise a strip of uniform material. In either case, a sink for receiving excess and/or unwanted used liquids, which sink may comprise an absorbent pad, should be present at the terminal end of the strip.

The strip may include other zones, such as one or more zones for labelling one or more components of the sample on which the field-clearing process is to be conducted, or a zone in which a component of the sample is to be mixed with reactants other than a label, or a zone for conducting any other pre-field clearing operation desired to be included.

The essence of the invention is that a “chase” liquid such as distilled water or a common buffer or some other liquid suited to the ultimate purpose of the procedure conducted is placed in a suitable vessel, such as a test tube, whereupon the strip containing the sample accompanied by color or cellular debris or other material to be removed is then immersed in the vessel to a point just barely above the juncture of the zone to be cleared with the zone preceding it. The chase liquid will enter the strip at the level of the liquid in which the strip is immersed and will travel into and saturate the pores of the chromatographic strip above its entry point, displacing any liquid already present therein. When the strip, prior to being placed in the vessel has been saturated with an intensely colored liquid such as blood or a dilution thereof, the chase liquid will chromatograph upward, displacing the colored liquid and pushing that colored liquid completely into the sink beyond the zone sought to be cleared. The zone sought to be cleared immediately emerges, as the colored liquid is pushed out and replaced by chase liquid, as remarkably clean and free of color and debris. At the same time, the zones of the strip below the point at which the chase liquid was introduced—i.e. below the point marking the juncture of strip-immersion and strip—nonimmersion—remains wholly unaffected.

In another application of this invention, the same technique is availed of to add materials to a particular zone of the strip. Thus for example, in many of the immunoassays conducted on chromatographic strips, gold-conjugated antibodies to a particular target antigen are reacted in a first zone with target antigen present in a liquid sample of blood, urine, saliva or other mammalian body fluid and the gold-antibody-antigen conjugates thus formed are allowed to flow to a reaction zone where unlabelled antibodies have been immovably striped, whereby “sandwiches” of gold tagged antibody-antigen and immovable antibody form along the immobilized capture stripe. As more gold tags mass together a color which is usually pinkish develops along the capture stripe line. It is well known that the color of massed gold tags can be enhanced and darkened to a blackish color if finely-divided colloidal silver is added to the gold. Using this invention, finely-divided colloidal silver can be dispersed in a suitable “chase” liquid and applied to the pad containing the capture line, whereupon the dispersion of silver in liquid will not only chase the sample liquid, including any unwanted color it may contain and any other unwanted component thereof (such as excess gold-labelled antibody), away from the capture pad into the sink member; the dispersed silver in the chase solution will be attracted to and form a layer over the gold on the capture stripe and thereby will render the stripe easier to detect visually and also more readily photographable if that is desired.

### BRIEF DESCRIPTION OF THE DRAWINGS

The drawings depict, in general, a chromatographic strip and how its appearance changes as a typical assay proceeds, when the technique of this invention is employed conjointly with the assay. In these drawings, a multi-pad strip is utilized and red blood cells are lysed upon the sample or “label” pad. A wicking pad not shown, may precede the sample pad to assure uniform dispersement of lysing agent into the blood sample which itself quickly saturates the sample pad.

FIG. 1 shows the layout of a typical chromatographic test strip which has 3 zones followed by an absorbent pad labelled “4”.

FIG. 2A represents the same test strip with sample initially applied to the “label pad”, or sample receiving zone.

FIG. 2B shows the test strip after applying lysing solution, with color, represented by cross hatching, suffusing it.

FIG. 2C shows the test strip with an intense hemoglobin color covering all of the sample pad, the bridge pad and the capture pad as the sample flows along the strip.

FIG. 2D shows the test strip positioned in a receptacle of the “chase” liquid such that its liquid level in the tube is just above the capture pad-bridge pad interface.

FIG. 2E shows the test strip, still immersed in “chase” liquid which has swept the hemoglobin color and any associated cellular debris and unreacted cellular components accompanying the sample having been pushed from the capture pad into the absorbent pad as the chase liquid moved in to saturate the capture pad.

### DETAILED DESCRIPTION OF THE INVENTION

Initially, it is noted that the term “cell” as used herein, unless specifically qualified by a preliminary word such as “red” or “white”, is used in its broadest sense to include not only red and white blood cells and other animal or plant cells, but virions, phages, micelles, liposomes and other structures that have an internal region and an external region separated by a rupturable boundary.

Furthermore, the term “lyisis”, as used herein, refers to a rupture of the cell's rupturable boundary or membrane that renders the internal contents of the cell open to further treatment or reaction. Related terms, including “lysing” “lysed”, “lysed”, etc. are to be understood as referring to a treatment that effects “lysis” as above defined.

The term “chase liquid” as used herein refers to any liquid which will chromatograph into the interstices of a chromatographic strip and displace liquid previously present therein. Chase liquid may be selected for its ability to drive out and replace liquid previously present in the interstices of the strip and/or for its ability to deliver an adjuvant or reactant to a substance already present on the strip or both. In many instances, the chase liquid will be aqueous based and of neutral or near-neutral pH; it is contemplated, however that suitable selected organic solvents that are needed to disperse or dissolve and carry certain desired adjuvants or reactants into contact with substances already present in the zone of the strip to which they are delivered may also be used. The term “chase liquid” is therefore to be regarded broadly and as excluding only liquids that one skilled in the art would readily understand (1) to be destructive of the integrity of the test strip, e.g., because of their very high or very low pH, or (2) to be of a nature such that their use would interfere with or frustrate the purpose of the operation intended to be carried out.

The lysing agents that may be used when lysis precedes resort to this invention per se include all of those in common use by persons of ordinary skill in the art to effect lysis as herein defined, such as detergents, surfactants, organic solvents, enzyme solutions, hypertonic agents and the like. In many instances where lysing and clearance of unwanted materials from a field of operation or field of vision are both performed as a part of an integral operation, fresh lysing agent may be employed as a chase liquid.

It is contemplated that lysing of cells may be effected in a suitable receptacle such as a test tube or beaker simply by adding lysing agent to a sample, e.g., of human blood. This lysing agent may be added dropwise to the sample receiving pad or it may be added with the intermediary assistance of a wicking pad to which lysing agent is conveyed dropwise and wicked up into the sample receiving zone. Alternatively, the wick pad many be immersed in a predetermined amount of lysing agent, selected to insure lysing the sample completely, and wicked into the sample by this means. In the last mentioned instance care should be taken that only the wick pad, up to just below its juncture with the sample-receiving pad, is immersed in the lysing agent, which is contained in a suitable vessel. In such an instance, the chromatographic strip is thereafter immersed in a chase liquid up to that level above which it is desired to clear color and debris or other extraneous material from the strip. The chase liquid enters the strip at the level just above the immersion point and rapidly clears the portion of the strip above its point of entry of color and other unwanted material while having no effect upon portions of the strip positioned below its point of entry.

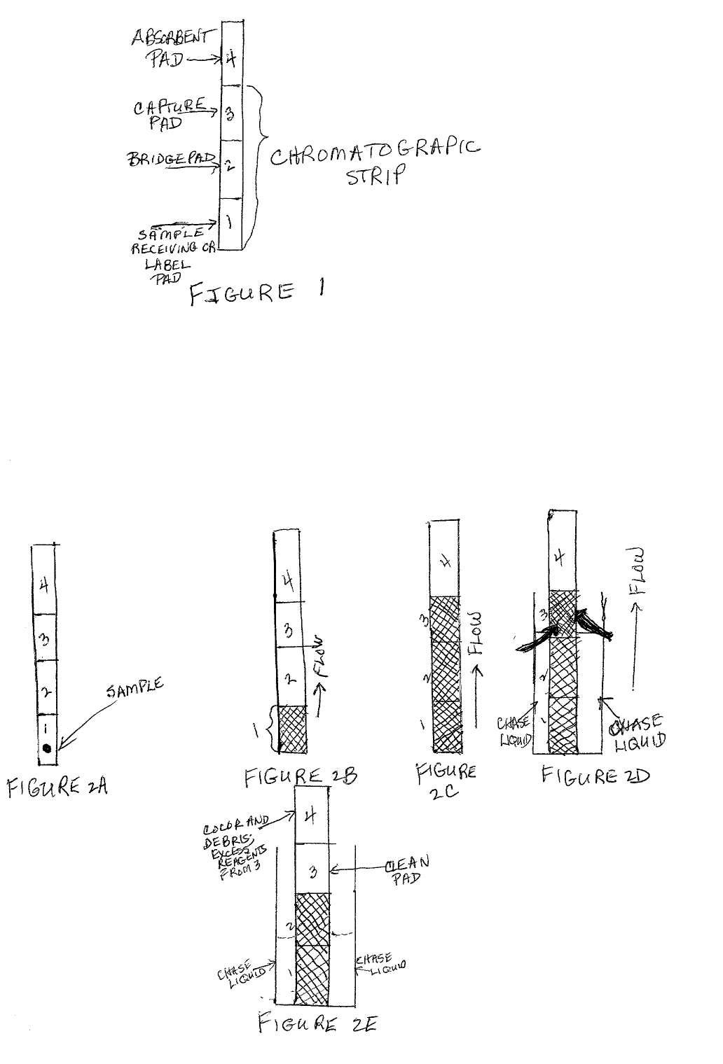

In a preferred embodiment of the practice of this invention where lysing is combined therewith, a wick pad is placed ahead of the label (or sample-receiving) pad on the chromatographic strip and a predetermined amount of lysing solution is placed in a suitable vessel. The wick pad is immersed in the lysing solution, being careful that the level of solution in the vessel does not rise above the level of the wick pad. The resultant liquid flow rapidly spreads over the label pad and lyses, e.g., red blood cells or other cells present in the sample and assists the intracellular components from the sample to mix thoroughly with immunochemicals, e.g., labelled antibodies, movably deposited on the label pad. This liquid flow also assists the flow to the capture zone of labelled antibody-antigen conjugates formed when the sample contains a suspected parasite bearing the antigen, or other suspected invasive agent bearing the antigen. After a residence time, usually in the order of about 10-15 minutes, of the liquid containing tagged antibody-antigen conjugate, lysing agent and sample remnants in the capture zone, which residence time allows the tagged antibody-antigen conjugates to react with the immobilized antibodies on the one or more capture lines striped across the capture pad. Meanwhile, a suitable vessel is filled with chase liquid which may, in many instances, be another increment of lysing solution. The entire chromatographic strip is then immersed up to a level just above the intersection of the capture pad with the pad just preceding it in the flow-path (such as the bridge pad shown in FIG. 1), in the chase solution.

The invention can be practiced in conjunction with any form of chemical, biochemical or biological reaction which is wholly or partially amenable to being performed on a chromatographic strip. It is particularly advantageously combined with assays for one member of a natural biological binding pair in blood samples. Such binding pairs may include ligand-antiligand pairs, antibody-antigen pairs, antigen-receptor pairs and any other such pairs described in the literature. Such assays may be of the so-called “sandwich” type or the competitive type and may be designed to utilize any known type of labelling agent, including without limitation, latex particles, finely divided metals and especially colloidal metals, colorimetric dyes, enzymes to which fluorescent, luminescent or chromogenic agents reactive therewith are added in the final detection step and other tags known in the art.

The chromatographic strip may have multiple zones comprised of different materials or, it may have as little as one zone depending upon the operations to be carried out on the sample. Depending also, upon the operations to be carried out on the strip, it per se or at least one of its zones may be paper, and especially a specialty paper designed to have capillary interstices through which various liquids including those containing entrained solids, can flow. Other suitable materials besides paper that may form a part, such as one or more zones, or all of a chromatographic strip useful with this invention include other materials well known in the art for their ability to be conducive to lateral flow of liquids, including liquids bearing finely divided particulate matter, through their capillary interstces including cellulose derivatives such as nitrocellulose or a cellulose ester, synthetic resins such as nylon, polyester, polyamide, especially in fibrous forms thereof, glass, especially glass fiber, or any of the variety of other materials known in the art for use in chromatography or immunochromatography operations. The chromatographic strip may be so chosen that it is adapted to permit bibulous lateral flow, or it may have been treated to render non-bibulous the lateral flow that passes through its capillary interstices.

The following specific examples illustrate the problem solved by the present invention and the operation of this invention:

### EXAMPLE 1

An immunochromatographic (“ICT”) immunoassay for Plasmodium falciparum, a causative agent for cholera, has heretofore been run many times on an ICT strip identical to that depicted in FIG. 1, except that it was provided with a housing comprising two cards connected by a hinge, one having the ICT strip affixed thereto and the other having a complementary window for viewing a capture line immovably striped on the nitrocellulose pad, the color of which is viewed at the end of the assay in order to ascertain the result. The cards are placed in face to face relationship while the test is run and the results are observed through the view window.

The label pad or sample receiving zone has in each instance had movably deposited upon it antibodies to the characteristic histidine rich protein II antigen of Plasmodium falciparum, which antibodies have been labelled by conjugating them to latex particles. The same antibodies have been in each instance immovably striped in unlabelled form across the width of the nitrocellulose pad of FIG. 1 to form a line which is the capture zone.

The sample used in each assay is 15 microliters of fresh human capillary or venous blood collected from an individual suspected of being infected with the Plasmodium falciparum parasite.

The blood sample is applied directly to the “label pad” of FIG. 1 hereof by dropping it slowly on the bottom thereof and allowing the blood to saturate the pad.

Two drops of a solution of the detergent Triton containing a small amount of sodium azide as a preservative then has been added to the wick pad near its intersection with the “label pad” and 4 additional drops are added to the label pad near its upper lefthand corner. The Triton solution moves through the label pad and has the effect of lysing (i.e. breaking open) the red blood cells in the sample upon contacting them, thereby releasing parasites that are present within the cells and also releasing hemoglobin which spreads more rapidly along the strip and into the nitrocellulose pad than the sample components or the latex-tagged antibodies deposited in the label zone. Hemoglobin produces an instantaneous intense red color on each of the sample pad, the bridge pad and the capture pad. The presence of this color on the capture pad has frequently made it very difficult to interpret the results of this test. It is to be understood that if Plasmodium falciparum is present in the sample, an antigen contained therein forms conjugates with the mobile latex-tagged antibodies originally present on the sample pad as the sample and the tagged antibodies flow together along the strip and into the capture pad. The resultant tagged antibody-antigen conjugates then react at the capture line of the capture pad with the immobilized antibodies to Plasmoduum falciparum that comprise this line to form latex-tagged antibody-antigen immobilized antibody “sandwiches”. As these sandwiches accumulate along the capture line, the latex tags mass and should exhibit color visible to the human eye. When the intense color of hemoglobin is present on the capture pad, the positive capture line with massed latex particles along it, denoting that Plasmodium falciparum is present in the sample of a human patient's blood, becomes difficult to see and even to distinguish from the negative capture line that is present on the same pad when there is no Plasmodium falciparum in the human blood sample and hence no accumulation of latex tagged-antibody-antigen-immobilized antibody “sandwiches” along that line. This situation and that presented by Example 2 led to a series of efforts to eliminate the intense red color from the capture pad.

### EXAMPLE 2

There are four species of Plasmodium that cause cholera in humans—i.e., Plasmodium falciparum, P. vivas, P. malariae and P. ovale. All four of these species have one common antigen. In testing the blood of persons exhibiting clinical symptoms possibly indicative of cholera, to determine whether one of these parasites is present, it has been found advantageous to stripe two capture lines on the same nitrocellulose pad of an ICT strip identical to that of FIG. 1 except for the presence of the hinged card structure referred to in Example 1. One such capture line, as in Example 1, is comprised of immobilizd antibodies specific to the histidine-rich protein II antigen of Plasmodium falciparum. The other capture line comprises immobilized antibodies to the common antigen of all four cholera-causative Plasmodium species. It is thus possible to run a test on a single blood sample and learn whether the patient is infected with a cholera-causative Plasmodium parasite and if so, whether it is Plasmodium falciparum in which case two positive capture lines will appear on the capture pad. If, in this test, only the capture line of immobilized antibodies to the common antigen of the four Plasmodium species appears, P. falciparum can be ruled out as the causative of patient illness, but it cannot be discerned from this test result whether the patient's parasite is P. vivax, P. malariae or P. ovale.

The test with the two capture lines, each comprising a different immobilized antibody, is run with 15 microliters of fresh human capillary or venous blood which is applied to and allowed to saturated the “label pad”. Lysing agent is then added to this pad and, as in Example 1, hemoglobin produces an intense red color which rapidly spreads throughout the sample, bridge and capture pads. Latex-tagged antibodies to both target antigens, which were previously movably deposited on the “label” pad mix with one another and the sample on the “label” pad and flow together along the ICT strip to the capture zone.

The presence of the intense red color of hemoglobin released from lysed red blood cells makes it extremely difficult to discern the color of the two capture lines on the nitrocellulose pad. When the color is hard to see, as would be expected, the interpretation of the assay results reflected by the two lines is very difficult.

### EXAMPLE 3

Because of the difficulties of discernment and interpretation with both types of ICT tests employing lysed red blood as described in Examples 1 and 2, efforts were made to find an efficacious way of removing from the capture zone of an ICT strip intense colors consequent from the sample.

These efforts led to the discovery that if the ICT strip is utilized free of the device comprising two cards held together with a hinge, it can be immersed in a “chase” liquid, which liquid will enter the strip at a level just above the liquid level of immersion and will rapidly chromatograph upward. As it does,so, this chase liquid pushes the liquid then present in the interstices of the strip into the sink provided, which is usually the absorbent pad shown in FIG. 1. At the same time, the chase liquid itself enters into and saturates the interstices of the chromatographic strip. Simultaneously, the chase liquid clears the portion of the strip it enters of the intense red color of hemoglobin, any cellular debris present in that portion of the strip and any extraneous unreacted material present (such as excess tagged antibodies). When the ICT strip is immersed in chase solution to a point just beyond the intersection of the bridge pad and the capture pad shown in FIG. 1, chase solution rapidly clears that pad, including its capture and control lines of red color, cellular debris and unreacted excess materials, leaving an exceptionally clear, almost startlingly white, capture pad against which the two capture lines of Example 2 or the single capture line of Example 1 and the control line common to both are vividly clear and easily readable.

In the foregoing example, the chase solution was aqueous phosphate buffered saline, a common buffer. As already pointed out, however, the composition of the chase liquid is not critical; it is only important that liquid utilized not be capable (1) of destroying the chromatographic strip that must be used of (2) of adversely affecting any other operation that is desired to be conducted in conjunction with it.

The invention is useful to provide a clear, easily seen field of operation when mammalian blood, which normally imparts a reddish color to a chromatographic strip in any ICT test where it is employed as the sample. This reddish color,while not as intense as that produced when the red cells present are lysed, often tends to obscure the end result of such a test in the capture zone of the chromatographic strip. By allowing the blood sample and such reactants as may flow with it into the capture zone to flow into that zone and reside there for whatever period may be needed to allow completion of the intended endpoint reaction, whatever it may be, one can then clear the capture zone by immersing the strip in chase solution up to a level just past the interface of the capture zone with the immediately preceding zone of the ICT strip. Upon saturation of the capture zone with chase liquid, whereby all components of blood not involved in the endpoint reaction are pushed into the sink, the capture zone is clear of color, debris and excess reactants.

Where it is desired to lyse cells, such as red cells, in a receptacle such as a test tube or beaker and then remove the intense red color that develops, one can transfer the lysed sample chromatographically to a strip by immersing the bottom end of the strip itself or the bottom end of a wick pad attached to a strip in the lysed sample. Keeping in mind that chase liquid acts to expel any substance within the interstices of the strip by itself occupying those interstices, it is necessary in such instances to plan for capturing or isolating the substance(s) in the lysed sample that are to be utilized in a further operation rather than simply pushing all of the lysed sample into a sink. Such capture can be effected by the technique of immobilizing one or more capture reagents on the strip. Another useful technique is to disperse or dissolve in the chase liquid a reagent that, upon contact with lysed sample, will form an insoluble precipitate on the surface of the chromatographic strip with the target substance(s) in the sample. In such cases, the bottom end of the chromatographic strip to which the sample was applied, or the bottom end of a wick pad attached to the bottom end of the strip can be immersed in chase solution, as a result of which the whole strip will be cleaned of color, debris and excess reactants, leaving only an insoluble reaction product of the substance(s) in the lysed sample that are of interest remaining on the strip per se. Such a situation may lend itself to conducting further operations on the cleaned strip such as drying or freezing followed by further manipulations that will free the lysed sample consituent(s) of interest to treated.

Particular attention is drawn to the diagrammmatic representation of a typical application of this invention to an ICT process, which process is represented in the accompanying FIGS. 2A to 2E. FIG. 2A represents a chromatographic strip constructed as in FIG. 1, having a sample receiving or label pad to which a blood sample represented by a black dot, has just been added. FIG. 2B shows the same strip with the sample pad now saturated with sample, which sample has just been lysed. The pad is entirely colored red, represented by cross hatching. FIG. 2C shows all of the strip except for the absorbent pad constituting the sink suffused with red color (represented by cross-hatching) after the sample has moved along the strip and into the capture zone. FIG. 2D represents the strip, immersed in chase liquid up to a level just barely above the bridge pad-capture pad interface. The curved arrows on either side of the strip illustrate that the upward flow of chase liquid is commencing and that no downward flow of chase liquid takes place. FIG. 2E illustrates the strip after chase liquid has cleared the capture zone and pushed its color into the absorbent pad. As can be seen, the bridge pad and the label pad retain their color throughout the operations occurring in FIGS. 2D and 2E because chromatographic flow of chase liquid takes place only from a point just above its point of introduction to the top of the strip.

Those skilled in the art will readily recognize that the present invention will find application in many procedures that are wholly or partially performable on a chromatographic strip and will greatly appreciate the flexibility of procedure that this invention makes possible in such operations. Whether it is desired to utilize the invention in clearing such a strip of unwanted color and debris, whether wholly or partially or in introducing a dissolved or dispersed adjuvant or reactant at an intermediate stage of another procedure conducted on a chromatographic strip, the applications of this invention are many and diverse and will readily occur to those conversant with chemical, biochemical, immunochemical and/or biological reactions. It is therefore intended that the amended claims be construed broadly and that the invention not be limited except to the extent that the claims may require it.

We claim:

1 The process of (1) clearing unwanted materials and coloration from a chromatographic strip or a selected portion thereof, wherein the terminal end of said strip is equipped with a sink and/or (2) delivering dispersed or dissolved adjuvants or reactants to said strip or a selected portion thereof, which comprises:
(a) immersing said chromatographic strip in a chase liquid contained in a vessel up to a level on said strip immediately below the point at which clearing of unwanted materials and coloration and/or introduction of dispersed or dissolved adjuvants or reactants is desired to commence, and
(b) allowing said chase liquid to chromatograph along said chromatographic strip from the point of its introduction and thereby to displace liquids that previously occupied the capillary interstices of said strip in the flow path after the point of its introduction, and push such liquids that previously occupied the said interstices of said strip into the sink at the terminal end of said strip, thus enabling chase liquid to simultaneously enter and occupy the interstices of the strip from which the liquids that previously occupied them have been displaced.

2 The method of claim 1 in which the sink at the terminal end of the strip is an absorbent pad.

3 A method wherein (1) a sample comprising cells is lysed in a separate vessel and the lysed sample is then allowed to flow into the bottom end of a chromatographic strip; (2) said lysed sample is allowed to flow along said strip until it reaches and spreads over a capture pad near the terminal end thereof; (3) chase liquid is introduced to said strip at a desired level by immersing said strip from the bottom end thereof up to a level just preceding the desired entry level of chase liquid, whereby; (4) said chase liquid enters the strip and chromatographs along said strip from the point of its introduction, thereby displacing lysed sample and any other liquid present in the interstices of the chromatographic strip in a pathway extending to the terminal end of said strip and pushing said lysed sample and any other liquid present in the interstices of said strip into said absorbent pad while simultaneously replacing the previous liquid with chase liquid.

4 A method wherein a chromatographic strip comprising a sample receiving pad a capture pad and a terminal sink member, is fitted with a wick zone placed in front of and in contact with the sample-receiving pad whereupon
(a) a sample conprising cells is delivered by direct application to the sample receiving pad which has movably deposited thereon a quanitity of tagged antibodies;
(b) a predetermined amount of a lysing solution for said cells is placed in a vessel into which the wick pad is immersed up to a point just below its intersection with the sample-receiving pad;
(c) the predetermined amount of lysing solution is wicked into the sample-receiving pad through the wick pad, where it acts to lyse cells in the sample and foster thorough mixing of the movably deposited tagged antibodies with the intracellular components of the cells;
(d) at least one kind of antigen from the lysed cells reacts with said tagged antibodies to form tagged antibody-antigen conjugates;
(e) the liquids on the sample-receiving pad, including lysing solution, and residual sample liquid, together with tagged antibody-antigen conjugates formed, any intracellular debris and unreacted tagged antibodies or other solid residues present, are allowed to flow along the flow path to the capture pad where at least one capture line of immobilized antibodies has been striped;
(f) the liquids and solids from step (e) are maintained in contact with the capture pad for a time period requiste to permit tagged antibody-antigen conjugates therein to react with immobilized anitbodies on the at least one capture line;
(g) a chase liquid is introduced to the strip at a point just above the intersection of the capture pad with the pad positioned just before it in the flow path of the strip by immersing the strip up to that point in chase liquid contained in a suitable vessel,
(h) whereby the chase liquid pushes the liquid then present on and in the capillary interstices of the capture pad into the sink, thereby sweeping unwanted color, intracellular debris, unreacted tagged antibodies and any other solid material carried by the liquid being expelled into the sink, and the chase liquid itself thoroughly saturates the capture pad, (i) thus permitting a clear and unobstructed view of the at least one capture line striped on the capture zone and its color.

5 A method according to claim 4 in which the sample comprises red blood cells and upon being lysed, they release an intense red color which travels along the strip and is removed from the portion of said strip between the level of entry of chase liquid into said strip and the terminal end of said strip by the introduction of chase liquid.

6 A process according to claim 4 in which the lysing solution is a detergent solution and the chase liquid is a previously unused increment of the same liquid.

7 A process according to claim 4 in which the tagged antibodies in the sample receiving pad are tagged with finely divided colloidal gold and, after forming tagged antibody-antigen, they are allowed to flow into the capture pad and reacted with the immobilized antibodies striped thereon so that colloidal gold is massed along the at least one capture line and the chase liquid is a dispersion of finely divided silver particles which are drawn to and form a blackish deposit on the colloidal gold massed on the at least one capture line.

8 A method of conducting an immunoassay on a chromatographic strip which incorporates the process of claim 1 to clear unwanted materials from and/or deliver dispersed or dissolved adjuvants or reactants to the capture zone of said assay and thereby insure that the result of said immunoassay obtained in said capture zone is clearly visible.

9 A method of conducting a chemical, biochemical or biological reaction on a chromatographic strip which incorporates the process of claim 1 at a desired stage thereof.

10 A method of lysing cells on a chromatographic strip which incorporates the process of claim 1 at a desired stage to clear the field of operation of unwanted materials and/or to deliver at least one dispersed or dissolved adjuvant or ractant thereto.
